(19) United States
(12) Patent Application Publication
(10) Pub. No.: US 2011/0053855 A1
(43) Pub. Date: Mar. 3, 2011

(54) Ex vivo cell stimulation

(75) Inventor: Reinhard Koenig, Rockville, MD (US)

(73) Assignee: NOVOGEN RESEARCH PTY LTD., North Ryde, New South Wales (AU)

(21) Appl. No.: 12/867,986

(22) PCT Filed: Feb. 19, 2009
(86) PCT No.: PCT/AU09/00187

§ 371 Date: Nov. 12, 2010

Related U.S. Application Data
(60) Provisional application No. 61/029,774, filed on Feb. 19, 2008.

(51) Int. Cl. A61K 38/39; C12Q 1/02; C12P 21/00; A61P 19/04; A61P 17/02

(52) U.S. Cl. 514/17.2; 435/29; 435/70.3

(57) ABSTRACT

Provided herein is are methods for evaluating the biological activity and/or therapeutic potential of a glucan, comprising in one embodiment: co-culturing a first population of cells with a second population of cells, wherein the first population comprises cells capable of being stimulated by said glucan to produce and/or secrete cytokines and growth factors, and the second population comprises collagen-producing cells; contacting said co-cultured cells with the glucan and incubating for a period of time sufficient to induce the production of collagen from the collagen-producing cells; and determining the level of production of collagen from said collagen-producing cells, wherein the level of production of collagen is indicative of the biological activity and/or therapeutic potential of the glucan.

### FIELD OF THE INVENTION

The present invention relates generally to methods for the ex vivo stimulation of cells by glucans. In particular, the invention relates to methods for stimulating macrophages and collagen-producing cells, such as fibroblasts, and for inducing collagen production from collagen-producing cells. Methods of the invention are applicable to, inter alia, evaluation of the biological activity and therapeutic potential of a glucan product, and the generation of cells and cellular products for administration to subjects in need of treatment.

### BACKGROUND OF THE INVENTION

Glucans are oligosaccharides or polysaccharides composed predominantly or wholly of glucose. Glucans are widely distributed in nature, being found in the cell walls of a variety of plants, fungi and microorganisms. Beta-(1,3)(1,6) glucans derived from yeast, such as the bakers yeast Saccharomyces cerevisiae, have been identified as having particular therapeutic potential for the treatment of a variety of disorders and conditions. Beta-glucans act to enhance the immune system, stimulating the activity of the primary defence cells, natural killer cells, neutrophils and macrophages. As such beta-glucans play a role in combating infection. Various beta-glucans have also been implicated in, for example, the treatment of cancer, septic shock, arthritis and in wound healing and reducing cholesterol.

A microparticulate beta-(1,3)(1,6) glucan from Saccharomyces cerevisiae, the isolation of which is described in U.S. Pat. No. 6,242,594, has been found to be therapeutically effective when administered, for example, to subjects suffering from a bone fracture, ulcers caused by physical trauma, surgical wounds, impaired blood flow, infections or neoplasia, or in persons in need of enhancement of fixation of implanted orthopaedic devices to bone. The glucan is also implicated in cosmetic skin surgery, tissue regeneration and tissue augmentation.

Accordingly, there is considerable interest in the development of pharmaceutical compositions comprising glucans.

A common problem facing the development of any composition designed for in vivo administration to a subject, in particular administration to humans, is the evaluation of the biological efficacy and therapeutic potential of the active ingredient(s). Biological efficacy and therapeutic potential can be adversely impacted by a number of factors. For example, many drugs and compositions are associated with side effects when administered to a subject. It is often difficult to ascertain the nature of such side effects in advance. Similarly, the therapeutic potential to be derived from any drug or composition can generally not be predicted with any confidence prior to administration. Quality assurance in the manufacturing process used to produce drugs and compositions for use on a clinical scale is of critical importance. There may be variability in activity between batches of any particular product and it is beneficial to have a reliable and simple means of detecting such variability.

Further to the above, it is becoming increasingly clear that the responses of different subjects to a particular drug or composition may differ, hence there is an increasing interest in so-called personalized medicine where treatments are tailored more specifically to the individual to be treated.

In view of such difficulties in the development of suitable therapeutics, there is a clear need for the development of simple and reliable methods for evaluating the biological activity and therapeutic potential of a drug or composition prior to its clinical application. For ethical reasons, it is apparent that such evaluations cannot be readily carried out on humans. There is also a significant trend against the use of animal models for such trials.

Currently used screening technologies for biological validation, pharmacological testing, and screening for success or failure of drugs and compositions in clinical trials typically suffer from a number of disadvantages including poor predictive value and absence of patient-specific focus.

Accordingly, there remains a clear need for the development of improved methods for the evaluation of biological activity and therapeutic potential. The present invention as disclosed herein provides suitable methods for evaluating the biological activity and therapeutic potential of glucans.

### SUMMARY OF THE INVENTION

The present invention is predicated, in part, on the inventor's finding that the in vitro stimulation of macrophages by a microparticulate glucan from yeast cells can be utilised to induce collagen production by fibroblasts. Thus, embodiments of the invention make use of intercellular communication between populations of different cell types to produce the desired effect, wherein only one of the cell populations is directly stimulated by the glucan. The stimulation of one cell population in turn provides other, non-stimulated cell populations with physiological signals initiating or enhancing their function.

According to a first aspect, the present invention provides a method for evaluating the biological activity and/or therapeutic potential of a glucan, the method comprising:
(a) co-culturing a first population of cells with a second population of cells, wherein the first population comprises cells capable of being stimulated by said glucan to produce and/or secrete cytokines and growth factors, and the second population comprises collagen-producing cells;
(b) contacting said co-cultured cells with the glucan and incubating for a period of time sufficient to induce the production of collagen from the collagen-producing cells; and
(c) determining the level of production of collagen from said collagen-producing cells,
   wherein the level of production of collagen is indicative of the biological activity and/or therapeutic potential of the glucan.

The populations of cells may comprise one or more cells. Typically the first population of cells comprises macrophages or precursors thereof. The precursors may be monocytes. The monocytes may be differentiated into macrophages prior to the co-culturing step. The collagen-producing cells may be, for example, fibroblasts or chondrocytes. The ratio of cells in the first population to cells in the second population may be between 1:50 and 50:1, optionally between 1:25 and 25:1, optionally between 1:10 and 10:1.

In a particular embodiment, the cells capable of being stimulated by the glucan are macrophages and the collagen-producing cells are fibroblasts.

The cells may be co-cultured in any suitable nutritive culture medium capable of sustaining both cell types. The culture medium may include additional co-factors for collagen production. The co-factors may be added to the culture medium either prior to, at the time of, or following addition of the glucan in step (b). Suitable co-factors include, but are not limited to, ascorbic acid and TGF-β1.

The glucan may be derived from any suitable cellular source, such as yeast cell walls. The glucan may be a particulate or microparticulate glucan, such as a microparticulate branched beta-(1,3)(1,6) glucan. The glucan may be microparticulate poly-(1,3)-beta-D-glucopyranosyl-(1,6)-beta-D-glucopyranose.

Typically the level of collagen production determined in accordance with the above aspect is compared with a predetermined control level of collagen production, which control comprises a co-culture of cells capable of being stimulated by the glucan and collagen-producing cells in the absence of the glucan, and whereby the difference in the level of collagen production is indicative of the biological activity and/or therapeutic potential of the glucan.

Collagen production may be determined by any suitable means, such as an enzyme-linked immunosorbent assay (ELISA).

According to a second aspect, the present invention provides a method for evaluating the biological activity and/or therapeutic potential of a glucan, the method comprising:
(a) mixing a first population of cells with a second population of cells, wherein the first population comprises cells capable of being stimulated by the glucan to produce and/or secrete cytokines and growth factors, and the second population comprises collagen-producing cells;
(b) co-culturing the first and second populations of cells for a period of time sufficient to induce the production of collagen from the collagen-producing cells; and
(c) determining the level of production of collagen from said collagen-producing cells,
   wherein the level of production of collagen is indicative of the therapeutic potential of the glucan, and wherein the first population of cells are incubated with the glucan prior to co-culturing step (b).

The glucan may be removed from the medium containing the first population of cells prior to the addition of the second population of cells. Alternatively, the glucan may remain in the medium in which the first and second cell populations are co-cultured.

In accordance with the above aspects, either or both of the first and second cell populations may be cultured or isolated from a subject. The cells of the first and second populations may be derived from the same or different individuals. The cells may be derived from a skin sample of the subject. The subject may be suffering from or be predisposed to a condition characterised by, or associated with, a collagen deficiency, collagen malfunction, or a connective tissue related condition benefiting from an augmentation of collagen in the connective tissue, including skin wounds or lesions, or a connective tissue disease or injury.

According to a third aspect, the present invention provides a method for predicting the response of a subject to administration of a glucan prior to said administration, the method comprising:
(a) co-culturing one or more cells capable of being stimulated by the glucan to produce and/or secrete cytokines and growth factors and one or more collagen-producing cells, wherein either or both of the cells capable of being stimulated by the glucan and the collagen-producing cells are isolated from a subject;
(b) contacting the co-cultured cells with the glucan for a period of time sufficient to induce the production of collagen from the collagen-producing cells; and
(c) determining the level of production of collagen from the collagen-producing cells,
   wherein the level of production of collagen is predictive of the response of the subject to administration of the glucan.

According to a fourth aspect, the present invention provides a method for predicting the response of a subject to administration of a glucan prior to said administration, the method comprising:
(a) mixing one or more cells capable of being stimulated by the glucan to produce and/or secrete cytokines and growth factors and one or more collagen-producing cells, wherein either or both of the cells capable of being stimulated by the glucan and the collagen-producing cells are isolated from a subject;
(b) co-culturing the cells for a period of time sufficient to induce the production of collagen from the collagen-producing cells; and
(c) determining the level of production of collagen from the collagen-producing cells,
   wherein the level of production of collagen is predictive of the response of the subject to administration of the glucan, and wherein the cells capable of being stimulated by the glucan are incubated with the glucan prior to co-culturing step (b).

Typically in accordance with the third and fourth aspects, the cells capable of being stimulated by the glucan to produce and/or secrete cytokines and growth factors and the collagen-producing cells are isolated from the same subject.

The cells capable of being stimulated by the glucan and/or the collagen-producing cells may be reintroduced into the subject from which the cells were isolated. Accordingly, the invention provides methods comprising such cells and compositions comprising the same.

According to a fifth aspect, the present invention provides a method for the ex vivo stimulation of collagen production, the method comprising co-incubating a first population of cells comprising cells capable of being stimulated by the glucan to produce and/or secrete cytokines and growth factors with a second population of cells comprising collagen-producing cells with an effective amount of a glucan for a time and under conditions suitable to stimulate the collagen-producing cells to produce collagen.

In a particular embodiment the glucan is a microparticulate glucan. The glucan may be derived from any suitable cellular source, such as yeast cell walls. Typically the glucan is a branched beta-(1,3)(1,6) glucan. More typically the glucan is microparticulate poly-(1,3)-beta-D-glucopyranosyl-(1,6)-beta-D-glucopyranose.

In a particular embodiment, the cells capable of being stimulated by the glucan are macrophages and the collagen-producing cells are fibroblasts.

The macrophages and fibroblasts may be isolated from a subject. The macrophages may be isolated in the form of monocytes. Typically the subject is a human. The subject may be suffering from or be predisposed to a condition characterised by, or associated with, a collagen deficiency or otherwise suffer from a skin wound or lesion, or a connective tissue disease or injury.

According to a sixth aspect, the present invention provides a method for the ex vivo stimulation of collagen production, the method comprising:
(a) mixing a first population of cells with a second population of cells, wherein the first population comprises cells capable of being stimulated by the glucan to produce and/or secrete cytokines and growth factors, and the second population comprises collagen-producing cells; and
(b) co-culturing the first and second populations of cells for a period of time sufficient to induce the production of collagen from the collagen-producing cells,
   wherein the first population of cells had been incubated with the glucan prior to co-culturing.

Collagen produced in accordance with the method of the fifth or sixth aspect may be administered to a subject in need thereof. The subject may be suffering from or be predisposed to a condition characterised by, or associated with, a collagen deficiency or otherwise suffer from a skin wound or lesion, or a connective tissue disease or injury. In a particular embodiment, where the co-cultured cells were isolated from the same subject, the collagen so produced ex vivo is administered to that subject.

Cells of the first and/or second populations may be introduced into a subject following co-culture of the cell populations. Typically the cells are introduced into the subject from which the cells were first isolated.

Thus, the present invention also provides pharmaceutical compositions comprising glucans tested in accordance with methods of the invention, pharmaceutical compositions comprising collagen produced in accordance with the method of the fourth or fifth aspect, and pharmaceutical compositions comprising cells of the first and/or second populations.

According to a seventh aspect, the present invention provides the use of a glucan for the ex vivo stimulation of collagen production according to a method of the fifth or sixth aspect.

Also provided is the use of a microparticulate beta-(1,3)(1,6) glucan for stimulating a cell population to produce and/or secrete a biologically active molecule, wherein the cell population is not directly contacted with the glucan.

### BRIEF DESCRIPTION OF THE DRAWINGS

The present invention will now be described, by way of non-limiting example only, with reference to the accompanying drawings.

FIG. 1. Microscopic image of neonatal human fibroblasts in culture with macrophages 48 hours after the initiation of co-culture. The fibroblasts were able to adhere to the substrate by utilizing the vacant substrate between the macrophages.

FIG. 2. Collagen production (mean optical density) from human neonatal fibroblasts (HDF) either cultured alone (left half of graph) or co-cultured with human macrophages (MO) (right half of graph) in the presence or absence of microparticulate beta-(1,3)(1,6) glucan (Glucoprime™, batches GP06.002 and GP002/98), ascorbic acid (AA) or TGF-β1 (TGF). Endotoxin was used as a positive control. Fibroblast cultures alone (left half) produced collagen, but the addition of glucan did not result in an increase in collagen production above non-stimulated control. Fibroblast+macrophage culture (right half) resulted in at least a 2-fold increase in collagen production in the presence of glucan compared to the non-stimulated control.

FIG. 3. Collagen production (C-terminal of type I collagen; ng/ml) as measured by enzyme-linked immunosorbent assay (ELISA) in co-cultures of macrophages and neonatal human fibroblasts in the presence of various glucans. Significant collagen production over background (absence of glucan; no simulation) was observed only in the Glucoprime™ IGP06.002 stimulated cultures. These results indicate that macrophages activated with Glucoprime™ stimulate collagen production in fibroblasts. The remaining glucans failed to generate significant levels of collagen production above background.

### DETAILED DESCRIPTION OF THE INVENTION

The articles “a” and “an” are used herein to refer to one or to more than one (i.e., to at least one) of the grammatical object of the article. By way of example, “an element” means one element or more than one element.

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

As used herein the term “effective amount” is used in two contexts, ex vivo and in vivo. In the ex vivo context, an “effective amount” refers to a suitable amount of glucan required to stimulate cells capable of being directly stimulated by glucans to a sufficient extent to result in collagen production by collagen-producing cells. The exact amount required will vary from case to case depending on factors such as the nature of the cells being co-cultured, the amount or concentration of cells cultured, the length of time in which the glucan is incubated with one or both of the cell types, the glucan being used and the form in which the glucan is presented. Thus, it is not possible to specify an exact “effective amount” However, for any given case, an appropriate “effective amount” may be determined by one of ordinary skill in the art using only routine experimentation. In the in vivo context, an “effective amount” includes within its meaning a non-toxic but sufficient amount or dose of an agent or compound to provide the desired therapeutic effect. The exact amount or dose required will vary from subject to subject depending on factors such as the species being treated, the age and general condition of the subject, the severity of the condition being treated, the particular agent being administered and the mode of administration and so forth. Thus, it is not possible to specify an exact “effective amount” However, for any given case, an appropriate “effective amount” may be determined by one of ordinary skill in the art using only routine experimentation.

As used herein the term “glucan” includes both a glucan molecule(s) in a purified form or (for example as an isolated molecule) and a glucan present in a composition or formulation. Thus, for the present purposes, the glucan may be associated with one or more additional components, which components are typically not active agents in their own right.

As used herein the term “isolated” means that the cell or cells in question have been removed from their host, and associated material (other cells or extracellular material) reduced or eliminated. Essentially, it means the object cell type is the predominant cell type present. However the “isolated” cell(s) need not be completely free of extraneous material or impurities provided the extraneous material or impurities do not prevent the ex vivo culturing of said cell(s).

The term “response” as used herein in the context of a subject's “response” refers to both clinical response and cellular response. That is, in accordance with the invention a subject's response to the administration of a glucan may be characterised by, or assessed in terms of, the clinical response of the subject, for example as determined by changes in any one or more symptoms of a condition suffered by the subject. Alternatively or in addition, the response of the subject may be assessed or measured at the cellular level, for example in terms of altered gene expression, or changes in the level of production and/or secretion of molecules such as signalling molecules or extracellular matrix constituents.

As used herein the term “subject” includes humans, primates, livestock animals (eg. sheep, pigs, cattle, horses, donkeys), laboratory test animals (eg. mice, rabbits, rats, guinea pigs), companion animals (eg. dogs, cats) and captive wild animals (eg. foxes, kangaroos, deer). Typically, the mammal is human or a laboratory test animal. Even more typically, the mammal is a human

The term “therapeutic potential” as used herein refers to the potential ability of a glucan to effect a “response” in a subject to which the glucan is administered. Typically the “therapeutic potential” of a glucan is associated with the biological activity of the glucan, which in turn may be determined by a number of means known to those skilled in the art including the ability of the glucan to stimulate the release of cytokines and/or growth factors from macrophages or to indirectly stimulate the production of collagen from fibroblasts. However, more broadly, the term “therapeutic potential” refers to the ability of the glucan to treat a particular condition in a subject. In this context, the term “treat” will be understood to have the meaning provided herein.

As used herein the terms “treating”, “treatment”, “preventing” and “prevention” refer to any and all uses which remedy a condition or symptoms, prevent the establishment of a condition or disease, or otherwise prevent, hinder, retard, or reverse the progression of a condition or disease or other undesirable symptoms in any way whatsoever. Thus the terms “treating” and “preventing” and the like are to be considered in their broadest context. For example, treatment does not necessarily imply that a patient is treated until total recovery.

Microparticulate beta-(1,3)(1,6) glucan derived from Saccharomyces cervisiae has been shown to stimulate human, cultured macrophages. However as disclosed herein, this glucan does not directly stimulate fibroblasts, cells required for collagen. However, it has been surprisingly found that the addition of S. cerevisiae-derived microparticulate beta-(1,3)(1,6) glucan to macrophages cultured together with fibroblasts resulted in activation of fibroblasts as evidenced by a nearly two-fold increase of collagen production. Without wishing to be bound by any one theory or mode of action, it is speculated that macrophages, upon glucan stimulation, initiate stimulation of fibroblasts by cell-to-cell contact or cytokine messaging. This indirect stimulation of fibroblasts via activated macrophages after glucan administration is considered to play an important role in tissue regeneration using physiologic mechanisms of interaction between cell populations involved in wound healing.

Accordingly, disclosed herein is the use of a microparticulate beta-(1,3)(1,6) glucan for stimulating a cell population to produce and/or secrete a biologically active molecule, wherein the cell population is not directly contacted with the glucan. The cell population may comprise collagen-producing cells such as fibroblasts and the biologically active molecule may be collagen.

The present invention provides, inter alia, methods for evaluating the biological activity and/or therapeutic potential of a glucan. The methods described and contemplated herein therefore play an important role in determining the clinical efficacy of glucan compositions for administration to subjects in need thereof, and those skilled in the art will appreciate that by evaluating the therapeutic potential and/or biological activity of a glucan prior to its administration, clinical response can be improved.

In one aspect, there is provided a method for evaluating the biological activity and/or therapeutic potential of a glucan, the method comprising:
(d) co-culturing a first population of cells with a second population of cells, wherein the first population comprises cells capable of being stimulated by said glucan to produce and/or secrete cytokines and growth factors, and the second population comprises collagen-producing cells;
(e) contacting said co-cultured cells with the glucan and incubating for a period of time sufficient to induce the production of collagen from the collagen-producing cells; and
(f) determining the level of production of collagen from said collagen-producing cells,
   wherein the level of production of collagen is indicative of the biological activity and/or therapeutic potential of the glucan.

Also provided is a method for evaluating the biological activity and/or therapeutic potential of a glucan, the method comprising:
(d) mixing a first population of cells with a second population of cells, wherein the first population comprises cells capable of being stimulated by the glucan to produce and/or secrete cytokines and growth factors, and the second population comprises collagen-producing cells;
(e) co-culturing the first and second populations of cells for a period of time sufficient to induce the production of collagen from the collagen-producing cells; and
(f) determining the level of production of collagen from said collagen-producing cells,
   wherein the level of production of collagen is indicative of the therapeutic potential of the glucan, and wherein the first population of cells had been incubated with the glucan prior to co-culturing.

The cells cultured in accordance with the present invention may be prepared by any suitable method known in the art. The cells may be immortal cell lines or, more typically, are derived (isolated) from individual subjects. Where cells are isolated from subjects, typically both the population of cells capable of being directly stimulated by glucan to produce and/or secrete cytokines and growth factors and the population of collagen-producing cells are isolated from the same subject. Each population of cells may comprise as few as one cell. The ratio of the first and second cell populations may be between about 1:10 to about 10:1, about 1:9 to about 9:1, about 1:8 to about 8:1, about 1:7 to about 7:1, about 1:6 to about 6:1, about 1:5 to about 5:1, about 1:4 to about 4:1, about 1:3 to about 3:1, about 1:2 to about 2:1, or about 1:1.

The nutritive medium in which the cells are co-cultured may be any suitable medium capable of sustaining both cell types. By way of example only, the medium may be RPMI medium or Dulbecco's modified Eagle medium. The medium may contain various supplements as desired or required for the particular cell types to be cultured. For example, the culture medium typically further comprises cofactors required for collagen production, such as ascorbic acid and TGF-β1.

Embodiments of the present invention contemplate both the incubation of glucan with the co-culturing populations of cells, or alternatively the pre-incubation of the first cell population (cells capable of being directly stimulated by the glucan to produce and/or secrete cytokines and growth factors) with the glucan prior to co-culturing of the two cell populations. In general the incubation time of the glucan with the co-culturing first and second cell populations is determined by the time required for the culture to respond to the glucan, specifically the time required to elicit a stimulation or induction of collagen production by the collagen-producing cells. Similarly, where the glucan is first incubated with the first cell population (prior to co-culture), the incubation time is determined by the time required to ‘activate’ or ‘prime’ the cells of the first population such that upon subsequent co-culturing with the second cell population the collagen-producing cells are stimulated or induced to produce collagen. In both cases, the incubation time in the presence of the glucan may be from several minutes to several days, for example from abut 10 minutes to 5 days, or from about 30 minutes to 5 days, from about 60 minutes to 4 days, from about 6 hours to 3 days, from about 12 hours to 2 days.

Similarly the co-culture time of the first and second cell populations, whether in the presence or absence of glucan, is typically determined by the time required to elicit a stimulation or induction of collagen production by the collagen-producing cells. The culture time may be from several minutes to several days, for example from abut 10 minutes to 5 days, or from about 30 minutes to 5 days, from about 60 minutes to 4 days, from about 6 hours to 3 days, from about 12 hours to 2 days. This culture time may be concomitant with or in addition to the incubation time in the presence of the glucan.

By way of example only, described herein is one method for the ex vivo stimulation of collagen production. As exemplified herein, macrophages and fibroblasts are co-cultured in suitable medium, for example RPMI medium for 48 hours. After 48 hours the medium is removed and replaced with fresh medium supplemented with glucan. The macrophages and fibroblasts are cultured in the presence of the glucan for 24 hours at which time ascorbic acid and TGF-β1 are added. Incubation is continued for a further 24 hours before supernatants are harvested (48 hours after the initial glucan addition).

The collagen may be of any type. In a particular embodiment, the collagen is type I collagen. Collagen production may be determined by any suitable means well known to those skilled in the art. Collagen production may be determined at the transcriptional level, for example determining the presence and/or amount of collagen mRNA transcripts. Alternatively or in addition, collagen production may be determined or the translational level, for example involving detection of the presence and/or amount of collagen polypeptides, precursors or derivatives thereof. Detection of collagen polypeptides, precursors or derivatives thereof may be via a number of means including immunological means such as enzyme-linked immunosorbent assays (ELISA), mass spectrography, or chromatography. Similarly, such methods for the detection of collagen polypeptides, precursors or derivatives thereof may be used to determine the level of collagen secretion from collagen-producing cells to the extracellular environment. Accordingly, the determination of collagen “production” includes within its scope the detection or quantification of collagen gene expression, collagen protein expression, post-transcriptional and post-translational modifications of collagen and/or or measurement of collagen secretion. The analysis of collagen production may be carried out directly in the culture medium and/or in the collagen-producing cells to determine the amount of non-secreted collagen.

Typically, for the purpose of evaluating the biological activity and/or therapeutic potential of a glucan, the analysis of collagen production following incubation of cells in the presence of glucan is compared to the level of collagen produced following incubation of the equivalent cells in the absence of the glucan. Thus methods of the invention may comprise a step of assessing the results of the analysis of collagen production in relation to a control.

Typically the glucan is a microparticulate glucan, more typically a microparticulate branched beta-(1,3)(1,6) glucan such as poly-(1,3)-beta-D-glucopyranosyl-(1,6)-beta-D-glucopyranose. The glucan may be a microparticulate glucan prepared in accordance with the process as described in U.S. Pat. No. 6,242,594 (Kelly; the disclosure of which is incorporated herein by reference in its entirety). However those skilled in the art will appreciate that the scope of the present invention is not limited thereto.

By way of example only, as disclosed herein, embodiments of the invention find application in glucan manufacturing processes as part of batch control and quality assurance procedures. That is, the bioassay methods of the invention may be used to evaluate the level of biological activity of any particular batch of glucan product, wherein the ability of the glucan product to stimulate monocytes and macrophages, as determined by the level of collagen produced by collagen-producing cells co-cultured with the monocytes or macrophages, is indicative of the biological activity (and hence the integrity) of the glucan product. Those skilled in the art will readily appreciate that such evaluation is of particular importance in screening and assessing the viability and efficacy of a product or composition destined for therapeutic administration. The viability and/or efficacy of a product or composition may decrease over time and may be affected by a number of factors including storage conditions (such as temperature, humidity), variability of components (both consumables and equipment) used in the glucan manufacturing process or composition preparation process, and modifications in the glucan manufacturing process or composition preparation process.

It will therefore also be appreciated by those skilled in the art that embodiments of the present invention may be utilised in assessing the effectiveness and suitability of glucan manufacturing processes and processes for the production of pharmaceutical compositions and formulations comprising the glucan. For example, it may be desirable to test a novel manufacturing process, to modify an existing manufacturing process, scale-up production or automate production. In these circumstances the present invention provides means for reliably determining the validity of the alterations and their impact on the activity of the glucan produced. Similarly, the invention provides means for the reliable determination of the effect of formulating a composition comprising a glucan, for example, the effect of various diluents, adjuvants, carriers or other ingredients on activity of the glucan, or the effect on glucan activity of formulating the composition for a particular mode or route of delivery.

Embodiments of the invention also find application in the evaluation of the biological activity of and the potential for therapeutic application of novel glucan molecules and formulations.

A further application of embodiments of the invention in which the ability of the glucan product to stimulate monocytes and macrophages, as determined by the level of collagen produced by collagen-producing cells co-cultured with the monocytes or macrophages may be utilised is in so-called “personalized medicine”. The term “personalized medicine” is used herein in its broadest context to refer to the tailoring of pharmaceutical compositions and medicines for particular individuals based on and taking into consideration knowledge of the individual's phenotype and/or genotype. Thus, in selecting a composition or medicine to be administered to any particular individual, use is made of information such as the individual's medical history, clinical data and/or the individual's genotype in an attempt to ensure that the composition or medicine is particularly suited to the individual at the time of administration. “Personalized medicine” has the potential to revolutionise the provision of healthcare, however to date little success has been achieved. Embodiments of the present invention provide novel avenues and approaches for the development and delivery of personalized medicine.

Accordingly, an aspect of the present invention provides a method for predicting a subject's response to the administration of a glucan, the method comprising:
(d) isolating from the subject one or more cells capable of being stimulated by the glucan to produce and/or secrete cytokines and growth factors;
(e) isolating from the subject one or more collagen-producing cells;
(f) co-culturing the cells from (a) and (b) for a period of time sufficient to induce the production of collagen from the collagen-producing cells; and
(g) determining the level of production of collagen from said collagen-producing cells,
   wherein the level of production of collagen is indicative of the response of the subject to administration of the glucan.

In accordance with the aspects and embodiments of the present invention the subject may be suffering from or be predisposed to a condition characterised by, or associated with, a collagen deficiency, collagen malfunction, or a connective tissue related condition benefiting from an augmentation of collagen in the connective tissue, including skin wounds or lesions, or a connective tissue disease or injury. Conditions characterised by, or associated with collagen deficiency include, but are not limited to conditions in which the biosynthesis, assembly, posttranslational modification and/or secretion of collagen is affected, often due to an underlying genetic defect. Such conditions include connective tissue disorders (e.g. collagenopathies), muscle disorders (myopathies), and disorders of basement membrane disorders. Various wounds may also be associated with collagen deficiency and/or benefit from collagen administration or stimulation. The treatment of such wounds is therefore contemplated herein and are encompassed by the present application. Such wounds include, but are not limited to surgical wounds, burn wounds, chronic ulcers, pressure sores, bed sores, diabetic ulcers, and other wounds requiring collagen for neo-formation of skin and wound closure, conditions requiring tissue augmentation for treatment such as urinary incontinence, cosmetic applications for the treatment of facial wrinkles and blemishes, ultraviolet light-induced skin damage and surgical application for the treatment of tissue defects such as following trauma or surgery.

Also provided by the present invention are methods for the ex vivo stimulation of collagen production.

In one aspect there is provided a method for the ex vivo stimulation of collagen production, the method comprising co-incubating a first population of cells comprising cells capable of being stimulated by the glucan to produce and/or secrete cytokines and growth factors with a second population of cells comprising collagen-producing cells with an effective amount of a glucan for a time and under conditions suitable to stimulate the collagen-producing cells to produce collagen.

In another aspect there is provided a method for the ex vivo stimulation of collagen production, the method comprising:
(c) mixing a first population of cells with a second population of cells, wherein the first population comprises cells capable of being stimulated by the glucan to produce and/or secrete cytokines and growth factors, and the second population comprises collagen-producing cells; and
(d) co-culturing the first and second populations of cells for a period of time sufficient to induce the production of collagen from the collagen-producing cells,
   wherein the first population of cells had been incubated with the glucan prior to co-culturing.

Also contemplated by the present invention are methods of treatment wherein glucan, collagen produced in accordance with aspects and embodiments of the invention and/or ex vivo cultured cells stimulated (directly or indirectly) with glucan in accordance with aspects and embodiments of the invention are administered to subjects in need thereof. Typically, where cells are administered such administrations are autologous, that is the cells are re-introduced into the subject from which the cells were isolated.

Agents (glucan, cells or collagen) may be administered in accordance with the present invention in the form of pharmaceutical compositions, which compositions may comprise one or more pharmaceutically acceptable carriers, excipients or diluents. Such compositions may be administered in any convenient or suitable route such as by parenteral, oral, nasal or topical routes. In circumstances where it is required that appropriate concentrations of the desired agent are delivered directly to the site in the body to be treated, administration may be regional rather than systemic. Regional administration provides the capability of delivering very high local concentrations of the desired agent to the required site and thus is suitable for achieving the desired therapeutic or preventative effect whilst avoiding exposure of other organs of the body to the compound and thereby potentially reducing side effects.

It will be understood that the specific dose level of a composition of the invention for any particular individual will depend upon a variety of factors including, for example, the activity of the specific agents employed, the age, body weight, general health and diet of the individual to be treated, the time of administration, rate of excretion, and combination with any other treatment or therapy. Single or multiple administrations can be carried out with dose levels and pattern being selected by the treating physician. A broad range of doses may be applicable. Considering a patient, for example, from about 0.1 mg to about 1 mg of agent may be administered per kilogram of body weight per day. Dosage regimens may be adjusted to provide the optimum therapeutic response. For example, several divided doses may be administered daily, weekly, monthly or other suitable time intervals or the dose may be proportionally reduced as indicated by the exigencies of the situation.

Generally, an effective dosage is expected to be in the range of about 0.0001 mg to about 1000 mg per kg body weight per 24 hours; typically, about 0.001 mg to about 750 mg per kg body weight per 24 hours; about 0.01 mg to about 500 mg per kg body weight per 24 hours; about 0.1 mg to about 500 mg per kg body weight per 24 hours; about 0.1 mg to about 250 mg per kg body weight per 24 hours; about 1.0 mg to about 250 mg per kg body weight per 24 hours. More typically, an effective dose range is expected to be in the range about 1.0 mg to about 200 mg per kg body weight per 24 hours; about 1.0 mg to about 100 mg per kg body weight per 24 hours; about 1.0 mg to about 50 mg per kg body weight per 24 hours; about 1.0 mg to about 25 mg per kg body weight per 24 hours; about 5.0 mg to about 50 mg per kg body weight per 24 hours; about 5.0 mg to about 20 mg per kg body weight per 24 hours; about 5.0 mg to about 15 mg per kg body weight per 24 hours.

The agent may be administered in the form of pharmaceutically acceptable nontoxic salts, such as acid addition salts or metal complexes, e.g. with zinc, iron or the like (which are considered as salts for purposes of this application). Illustrative of such acid addition salts are hydrochloride, hydrobromide, sulphate, phosphate, maleate, acetate, citrate, benzoate, succinate, malate, ascorbate, tartrate and the like. Examples of pharmaceutically acceptable carriers or diluents are demineralised or distilled water; saline solution; vegetable based oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly. Typically, the carrier or carriers will form from 10% to 99.9% by weight of the compositions.

Topical formulations typically comprise an active ingredient together with one or more acceptable carriers, and optionally any other therapeutic ingredients. Formulations suitable for topical administration include liquid or semi-liquid preparations suitable for penetration through the skin to the site of where treatment is required, such as liniments, lotions, creams, ointments or pastes, and drops suitable for administration to the eye, ear or nose.

Lotions according to the present invention include those suitable for application to the skin or eye. An eye lotion may comprise a sterile aqueous solution optionally containing a bactericide and may be prepared by methods similar to those described above in relation to the preparation of drops. Lotions or liniments for application to the skin may also include an agent to hasten drying and to cool the skin, such as an alcohol or acetone, and/or a moisturiser such as glycerol, or oil such as castor oil or arachis oil.

Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol or macrogols.

Pharmaceutical forms suitable for injectable use include sterile aqueous solutions (where water soluble) or dispersions and sterile powders for the extemporaneous preparation of sterile injectable solutions. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The preventions of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilisation. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredients are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions in such that a suitable dosage will be obtained. Preferred compositions or preparations according to the present invention are prepared so that an oral dosage unit form contains between about 0.1 μg and 2000 mg of active compound.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

The present invention contemplates combination therapies, wherein agents as described herein are coadministered with other suitable agents which may facilitate the desired therapeutic or prophylactic outcome. For example, in the context of asthma, one may seek to maintain ongoing anti-inflammatory therapies in order to control the incidence of inflammation whilst employing agents in accordance with embodiments of the present invention. By “coadministered” is meant simultaneous administration in the same formulation or in two different formulations via the same or different routes or sequential administration by the same or different routes. By “sequential” administration is meant a time difference of from seconds, minutes, hours or days between the administration of the two types of molecules. These molecules may be administered in any order.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present invention will now be described with reference to the following specific examples, which should not be construed as in any way limiting the scope of the invention.

### EXAMPLES

### Example 1

### Stimulation of Collagen Production by Fibroblasts

Monocytes from human blood were isolated and differentiated into macrophages (MØ) and activated by yeast microparticulate beta-(1,3)(1,6) (Glucoprime™ as described in U.S. Pat. No. 6,242,594, the disclosure of which is incorporated herein) and other glucans. Neonatal Human Fibroblasts (NHDF) were introduced to the MØ population to determine if MØ cells activated by Glucoprime™ stimulated collagen production in NHDF.

Preparation of 4 mM HCl with 0.1% Bovine Serum Albumin

1.0 mL of 12N Hydrochloric Acid (HCl) (Sigma H41758) was added to 11.0 mL of Water for Cell Culture Applications (Cambrex 17-724Q) resulting in an intermediate stock of 1.0M. The intermediate stock was further diluted by adding 0.06 mL of the stock to 14.94 mL of Water for Cell Culture Applications for a final concentration of 4 mM. 0.015 g of Bovine Serum Albumin (BSA) (Sigma A-7906) was added to the 15.0 mL of 4 mM HCl for a final concentration of 0.1% of BSA in 4 mM HCl.

Preparation of 10 μg/mL Transforming Growth Factor Beta 1 (Human Platelet Derived) (hTGF-β1)

100μL of 4 mM HCl with 0.1% Bovine Serum Albumin was added to a 1.0 μg vial of hTGF-β1 (R&D Systems 100-B). The vial was then mixed via vortex for approximately 45 seconds. The 10 μg/mL stock was divided into 10 μL aliquots and stored frozen at −30° C.

### Medium Preparation

Complete MØ Medium was prepared with 450 mL of RPMI 1640 (Cambrex 12-167F) supplemented with 50.0 mL of Human Sera Type AB (Cambrex 14-498E), 5.0 mL of 200 mM L-Glutamine (Cambrex 17-605E) and 5.0 mL of Penicillin Streptomycin (Cambrex 17-602E).

### Preparation of 10 mM Ascorbic Acid

Approximately 0.2737 g of Ascorbic Acid (Sigma A8960) was weighed using a balance. 94.66 mL of Complete MØ Medium was added for a final concentration of 10 mM. The 10 mM AA stock was divided into 1.0 mL aliquots and stored at −30° C.

### Blood Collection and Processing

Human blood was collected from normal paid volunteers under informed consent via venipuncture into BD Vacutainer® CPT™ Cell Preparation Tubes containing Sodium Citrate (BD 362760). The identity of the donors was kept confidential by coding the blood samples. The tubes were centrifuged at 1800×g for 20 minutes at room temperature. Following centrifugation the tubes were inverted 5-10 times. Granulocytes and red blood cells had been pulled through the gel plug and the mononuclear cells, plasma, and platelets remain above the plug as a clear yellow suspension. The entire contents of the tube above the gel was distributed evenly into two 50 mL conical tubes using a 10 mL serological pipette. Phosphate Buffered Saline (PBS) (w/o Ca and Mg) (Cambrex 17-516Q) was added to bring the volume of each conical tube up to 50 mL. The tubes were centrifuged at 300×g for 10 minutes at room temperature. After centrifugation the supernatant was aspirated leaving approximately 5 mL in each tube. The cell pellets were resuspended and combined, rinsing the tube several times with PBS to ensure all of the cells were collected. Again, the volume of the tube was brought up to 50 mL with PBS and centrifuged at 300×g for 10 minutes at room temperature. Following centrifugation the supernatant was carefully aspirated leaving approximately 5 mL of PBS. The total volume was brought up to 10 mL using Complete MØ Medium and a cell count was performed, at a 1:50 dilution with Trypan Blue (Cambrex 17-942E).

### Cell Seeding and NHDF Addition

The counted peripheral blood monoculear cells (PBMCs) were seeded in 1.0 mL volumes at a concentration of 2.0×10⁶ cells per well in 24 well plates. The cells were incubated at 37° C. with 5% CO₂ for 12 days. The cultures were maintained by removing 800 μL of the spent medium and replacing it with 800 μL of Complete MØ Medium twice per week. On day 12 the non-adherent cells from the cultures were removed by first mixing the medium from each well with a 1.0 mL pipette and aspirating all of the medium in each well. 1.0 mL of PBS was added to each well and again mixed with a 1.0 mL pipette. The PBS was removed and replaced with 900 μL of Complete MØ Medium.

NHDF cells were added directly to the MØ cultures by quickly thawing 2-3 passage 4 amps of NHDF cells (Cambrex) in a 37° C. water bath. The contents of the amps were transferred into a 50 mL conical tube containing 5 mL of Complete MØ Medium. The amps were rinsed twice with 1.0 mL of Complete MØ Medium. The rinse was transferred into the 50 mL conical tube. Complete MØ Medium was added to the conical tube to reach a final volume of 25 mL. The conical tube containing the cell suspension was centrifuged at 210×g for 5 minutes at room temperature. Following centrifugation the supernatant was carefully aspirated without disturbing the cell pellet. The NHDF cells were resuspended in Complete MØ Medium and a cell count was performed using Trypan Blue. The NHDF cells were added directly to the MØ cultures in 100 μL volumes at a concentration of 6.0×10⁵ cells/mL (6.0×10⁴ cells/well) The cultures were incubated at 37° C. with 5% CO₂ for 48 hours. After 48 hours all of the medium was aspirated from each well and replaced with 900 μL of RPMI 1640 supplemented with 1.0% mL of Human Sera Type AB, 2 mM L-Glutamine and 1.0% of Penicillin Streptomycin.

### Glucoprime™ Preparation

Glucoprime™ suspensions were prepared in 1.0 mg/mL (10×) stock solutions by using a balance to weigh out the material and adding the appropriate amount of PBS to reach the desired concentration.

### Commercial Glucan Preparations

Glucan from Baker's Yeast (Sigma G5011), Laminarin (Sigma L9634), Dextranase (Sigma D8144), and Laminarinase (Sigma L9259) suspensions were prepared in 1.0 mg/mL (10×) stock solutions adding the appropriate amount of PBS to reach the desired concentration.

### Glucoprime™/Commercial Glucan Addition

100 μL of each lot of Glucoprime™ (IGP06.002 and IGP002/98) and each commercial glucan was added to 1× sample well of the MØ+NHDF cultures for a final concentration of 100 μg/mL. Vehicle control wells containing cells with 10% PBS (representing the concentration of PBS added to each culture with the Glucoprime™ or commercial glucan) and Complete MØ Medium were included on each plate as well as a positive stimulatory control of 10 ng/mL of Endotoxin (Cambrex N185) diluted in PBS. The cultures were incubated at 37° C. with 5% CO₂ for 24 hours. Following the 24-hour incubation Ascorbic Acid was added to each culture at a concentration of 100 μM and hTGF-β1 was added at a concentration of 5 ng/mL. The cultures were incubated at 37° C. with 5% CO₂ for 24 hours. Following the incubation the supernatants from each of the culture wells were harvested and diluted 1:5 in a polypropylene plate. The plate was centrifuged at 850×g for 10 minutes at room temperature to remove debris. The contents of the plate were transferred using a multichannel pipette into triplicate polypropylene plates and stored at −30° C. for future ELISA analysis.

### Metra®CICP EIA Assay

Collagen production by NHDF cells was quantified using a MetraCICP EIA kit (Quidel Corporation). The stored supernatants were thawed at room temperature for approximately 1-1.5 hours. The samples were further diluted using CICP Assay Buffer (see Attachment 1) 1:300 before being added to the assay plate. The MetraCICP Assay was performed according to the manufacturer's instructions.

### RESULTS

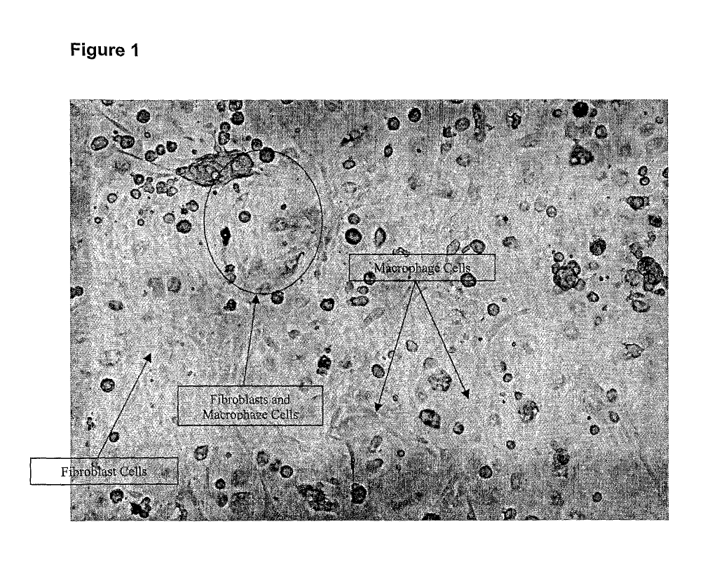

FIG. 1 is a light micrograph showing macrophages and fibroblasts in co-culture with secreted collagen.

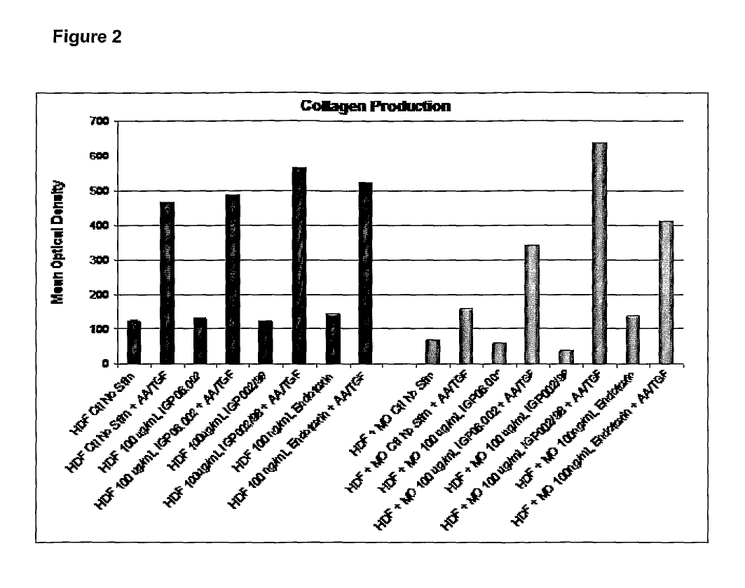

As shown in FIG. 2, fibroblasts cultured in the absence of macrophages produced collagen upon addition of co-factors ascorbic acid and TGF-β1. However the addition of Glucoprime™ did not result in an increase in collagen production above that observed in the absence of Glucoprime™. In contrast, fibroblasts co-cultured with macrophages resulted in at least a 2-fold increase in collagen production in the presence of Glucoprime™ (in the presence of ascorbic acid and TGF-β1) when compared to the co-culturing of fibroblasts and macrophages in the absence of Glucoprime™. Macrophages cultured in the absence of fibroblasts did not result in any collagen production (data not shown).

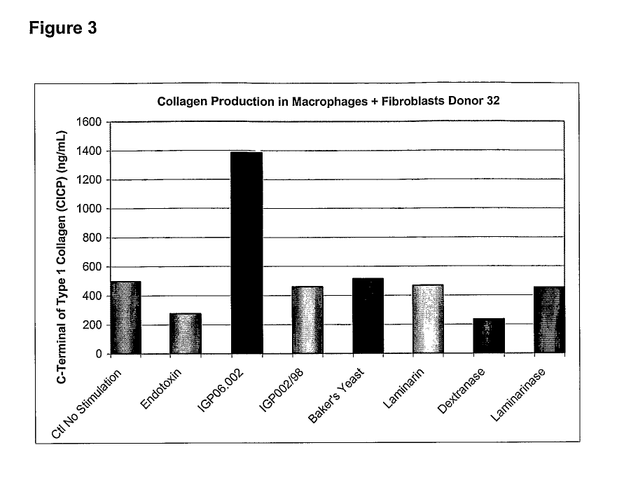

FIG. 3 shows collagen production (C-terminal of type I collagen) as measured by enzyme-linked immunosorbent assay (ELISA) in co-cultures of macrophages and neonatal human fibroblasts in the presence of various glucans, including Glucoprime™. Significant collagen production over background (i.e. in the absence of glucan) was observed only in the Glucoprime™ IGP06.002 stimulated cultures. These results indicate that macrophages activated with Glucoprime™ stimulate collagen production in fibroblasts. The remaining glucans failed to generate significant levels of collagen production above background.

1. A method for evaluating the biological activity and/or therapeutic potential of a glucan, the method comprising:
(a) mixing a first population of cells with a second population of cells, wherein the first population comprises cells capable of being stimulated by said glucan to produce and/or secrete cytokines and growth factors, and the second population comprises collagen-producing cells;
(b) mixing a first population of cells with a second population of cells, wherein the first population comprises cells capable of being stimulated by said glucan to produce and/or secrete cytokines and growth factors, and the second population comprises collagen-producing cells;
(c) determining the level of production of collagen from said collagen-producing cells,
wherein the level of production of collagen is indicative of the biological activity and/or therapeutic potential of the glucan, and wherein (i) the first population of cells is incubated with the glucan prior to co-culturing step (b), or (ii) the glucan is incubated with the mixed populations of cells during or immediately prior to co-culturing step (b).

2. (canceled)

3. The method of claim 1 wherein the first population of cells comprises macrophages or precursors thereof.

4. (canceled)

5. The method of claim 3 wherein the precursors are monocytes that are differentiated into macrophages prior to the co-culturing step.

6. The method of claim 1 wherein the collagen-producing cells are fibroblasts or chondrocytes.

7. (canceled)

8. The method of claim 1 wherein the cells are co-cultured in a suitable nutritive culture medium capable of sustaining both cell types, optionally including additional co-factors for collagen production selected from ascorbic acid and/or TGF-β1.

9-10. (canceled)

11. The method of claim 1 wherein the glucan is derived from yeast cell walls.

12. The method of claim 1 wherein the glucan is a particulate or microparticulate glucan

13. The method of claim 1 wherein the glucan is a microparticulate branched beta-(1,3)(1,6) glucan.

14. (canceled)

15. The method of claim 1 wherein the level of collagen production determined is compared with a predetermined control level of collagen production, which control comprises a co-culture of cells capable of being stimulated by the glucan and collagen-producing cells in the absence of the glucan, and whereby the difference in the level of collagen production is indicative of the biological activity and/or therapeutic potential of the glucan.

16. (canceled)

17. The method of claim 1 wherein the glucan is removed from the medium containing the first population of cells prior to the addition of the second population of cells.

18. (canceled)

19. The method of claim 1 wherein either or both of the first and second cell populations are cultured or isolated from a subject.

20. The method of claim 19 wherein the cells of the first and second populations are be derived from the same or different individuals.

21. The method of claim 20 wherein the cells are derived from a skin sample of the subject.

22. The method of claim 21 wherein the subject is suffering from or is predisposed to a condition characterised by, or associated with, a collagen deficiency, or otherwise suffer from a skin wound or lesion, or a connective tissue disease or injury.

23. (canceled)

24. A method for predicting the response of a subject to administration of a glucan prior to said administration, the method comprising:
(a) mixing one or more cells capable of being stimulated by the glucan to produce and/or secrete cytokines and growth factors and one or more collagen-producing cells, wherein either or both of the cells capable of being stimulated by the glucan and the collagen-producing cells are isolated from a subject;
(b) co-culturing the cells for a period of time sufficient to induce the production of collagen from the collagen-producing cells; and
(c) wherein the level of production of collagen is predictive of the response of the subject to administration of the glucan, and wherein (i) the cells capable of being stimulated by the glucan are incubated with the glucan prior to co-culturing step (b), or (ii) the glucan is incubated with the mixed cells during or immediately prior to co-culturing step (b).

25. The method of claim 24 wherein the cells capable of being stimulated by the glucan to produce and/or secrete cytokines and growth factors and the collagen-producing cells are isolated from the same subject.

26. The method of claim 25 wherein the cells capable of being stimulated by the glucan and/or the collagen-producing cells are reintroduced into the subject from which the cells were isolated.

27. A method for the ex vivo stimulation of collagen production, the method comprising co-incubating a first population of cells comprising cells capable of being stimulated by the glucan to produce and/or secrete cytokines and growth factors, and a second population of cells comprising collagen-producing cells, with an effective amount of a glucan for a time and under culture conditions suitable to stimulate the collagen-producing cells to produce collagen.

28. The method of claim 27 wherein the cells capable of being stimulated by the glucan are macrophages and the collagen-producing cells are fibroblasts.

29. The method of claim 27 wherein
the first population of cells is incubated with the glucan prior to co-incubating with the second population of cells under culturing conditions.

30. A method for the treatment of a subject suffering from or be predisposed to a condition characterised by, or associated with, a collagen deficiency or otherwise suffer from a skin wound or lesion, or a connective tissue disease or injury, the method comprising administering to the subject an effective amount of collagen produced in accordance with claim 27.

31-33. (canceled)
